(19) United States
(12) Patent Application Publication
(10) Pub. No.: US 2026/0335027 A1
(43) Pub. Date: Oct. 29, 2026

(54) Dynamic Reference Arrays and Methods of Use

(71) Applicant: GLOBUS MEDICAL, INC., AUDUBON, PA (US)

(72) Inventors: Nicholas Theodore, Ruxton, MD (US); Neil R. Crawford, Chandler, AZ (US); Mitchell A. Foster, Scottsdale, AZ (US)

(21) Appl. No.: 19/736,220

(22) Filed: Jul. 9, 2026

Related U.S. Application Data
(62) Division of application No. 17/471,315, filed on Sep. 10, 2021, now Pat. No. 12,714,507.
Application No. 17/471,315 is a division of application No. 14/602,723, filed on Jan. 22, 2015, now Pat. No. 11,116,576.
Application No. 14/602,723 is a continuation-in-part of application No. 13/924,505, filed on Jun. 21, 2013, now Pat. No. 9,782,229.
(60) Provisional application No. 61/800,527, filed on Mar. 15, 2013.
(60) Provisional application No. 61/662,702, filed on Jun. 21, 2012.

(51) Int. Cl. A61B 34/20; A61B 90/00; A61B 6/03; A61B 6/12; A61B 17/17; A61B 17/70; A61B 34/10; A61B 34/30; A61B 90/14

(52) U.S. Cl. CPC A61B 34/20; A61B 90/39; A61B 6/032; A61B 6/12; A61B 17/1757; A61B 17/7047; A61B 2034/107; A61B 2034/2051; A61B 2034/2055; A61B 34/30; A61B 90/14; A61B 2090/363; A61B 2090/374; A61B 2090/378; A61B 2090/3937; A61B 2090/3966; A61B 2090/3983; A61B 2090/3991; A61B 2090/3995

(57) ABSTRACT

Dynamic reference arrays use markers and trackers to register a patient’s anatomy to computer system. Wherein the dynamic reference array may be screwed into a patient’s spinous process, clamped on to a spinous process, or attached to the spinous process using posts. In embodiments, a dynamic reference array may comprise a single structure comprising and attachment member and a scaffold. In alternate embodiments, the dynamic reference array may comprise distinct structures that allow the dynamic reference array to swivel and collapse in order to facilitate registration, while not interfering with a surgical procedure.

### CROSS-REFERENCE TO RELATED APPLICATIONS

This application is a divisional application of U.S. Patent Application Serial No. 17/471,315 filed on Sep. 10, 2021, which is a divisional application of U.S. Application No. 14/602,723, filed on Jan. 22, 2015, which is a continuation-in-part of U.S. Application No. 13/924,505, entitled “Surgical Robot Platform,” filed on Jun. 21, 2013, which claims priority to U.S. Provisional Patent Application No. 61/800,527 filed on Mar.15, 2013 and to U.S. Provisional Patent Application No. 61/662,702 filed on Jun. 21, 2012, all of which are incorporated herein by reference in their entirety.

### BACKGROUND

Embodiments are directed to surgical navigation procedures and, more particularly, embodiments are directed to dynamic reference arrays, which include a plurality of active members, where the active members may be radio-opaque fiducial markers and tracking markers.

Dynamic reference arrays may typically be rigid bodies that may be temporarily attached to a patient’s anatomy during a navigated surgical procedures. Dynamic reference arrays may also commonly be referred to as “targeting fixtures.” Dynamic reference arrays may comprise marker trees, which may be attached to the patient’s anatomy using pins or clamps to the bone. A dynamic reference array’s purpose is to allow real-time tracking of the 3D position of the patient’s anatomy and mapping a patient’s anatomy to a computerized three-dimensional (3D) coordinate system. When a dynamic reference array is registered, it is possible for a mechanical system such as a robot to target a location on the patient’s anatomy corresponding to a known location in the computerized 3D coordinate system, enabling the mechanical system to guide insertion of a screw or needle, or perform another surgical or therapeutic procedure requiring targeting. Registering dynamic reference arrays may be done using point-to-point registration, point cloud registration, or alternative methods.

Point-to-point registration requires common, known points in the camera coordinate system and the anatomical coordinate system to be identified. The anatomical landmarks or reference marks on a feature of the dynamic reference arrays may be identified using a digitizing probe, which may be a wand with embedded tracking markers that enable the system to extrapolate the 3D location of the wand’s tip based on positions of the fiducial markers embedded in the wand’s handle. As an example, an image guidance system may indicate to the user through a software feature that the tip of the spinous process of L4 should now be touched by the wand, and the user may physically touch that point with the tool while confirming this procedure on software. Then the system may indicate that the tip of the spinous process of L5 should be touched by the wand, then other points, with the process repeated until enough points are identified to ensure good co-registration of the anatomical and camera coordinate systems.

Point cloud registration may typically require an array (cloud) of points to be identified manually by the user, typically by dragging the tip of a digitizing probe or wand across the surface of bone. After the bony contours may be characterized, the system may search the anatomical image for a matching bone surface contour. If the contour of the physically identified point array matches a contour found through image processing of the bony anatomy, then the anatomical coordinate system and the camera coordinate system may then be co-registered accurately. The methods of point-to-point registration and point cloud registration are known in the art.

In addition to using the methods of point-to-point registration and point cloud registration, an alternative method of automatic registration may be used that makes use of an additional piece that is temporarily or permanently mounted on the dynamic reference arrays. This registration method may automatically or manually locate in software the positions in the 3D medical image volume of three or more radio-opaque fiducial markers, referred to as a “fiducial array”. Radio-opaque refers to the property that the fiducial markers are visible and distinguishable in the 3D volume of the medical image. The physical positions of these fiducial markers may be found from the optical tracking system without user intervention because the fiducial markers may be mounted to a dynamic reference array in a known position relative to the tracking markers that are part of the dynamic reference array. Using this fixed relationship of fiducial to tracking markers, the known positions of the fiducial markers in the 3D image volume, and the detected positions of the tracking markers in the camera coordinate system, co-registration of the camera and image coordinate systems is possible.

It is preferable to mount the fiducial array near the location on the patient at which surgery is to be performed because the accuracy of localizing the anatomy decreases with increasing distance from the fiducials. However, it is preferable to mount the tracking markers away from the location on the patient at which surgery is to be performed so that the tracking markers do not interfere with positioning of surgical tools, retractors, etc.

Consequently, there is a need for a device that may use both radio-opaque fiducials and tracking members on the same dynamic reference array. Further, there is a need for positioning the dynamic reference array as close as possible the patient’s anatomy, while not interfering with surgical tools during a surgery. The ability to perform operations on a patient with a single dynamic reference array greatly diminish the time consumed in preparation for surgery and during surgery. The application of the dynamic reference array and the techniques used with the dynamic reference array may enhance the overall surgical operation and the results of the operation.

These and other needs in the art are addressed in one embodiment wherein a dynamic reference array may comprise a scaffold, a plurality of markers coupled to the scaffold, and an attachment member coupled to the scaffold with a swivel feature, wherein the attachment member is configured to secure the dynamic reference array to a patient while allowing movement of the scaffold with respect to the attachment member. In other embodiments, a method is addressed using a dynamic reference array that may comprise attaching the dynamic reference array to a patient, scanning the patient while a scaffold on the dynamic reference array is in a first position, registering the dynamic reference array to the patient’s anatomy, and moving the scaffold into a while the dynamic reference array remains attached to the patient.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other embodiments for carrying out the same purposes of the present invention. It should also be realized by those skilled in the art that such equivalent embodiments do not depart from the spirit and scope of the invention as set forth in the appended claims.

### BRIEF DESCRIPTION OF THE DRAWINGS

For a detailed description of the preferred embodiments of the invention, reference will now be made to the accompanying drawings in which:

FIG. 1 illustrates an embodiment of a dynamic tracking device mounted to the spinous process of the lumbar spine of a human spine model;

FIG. 2 illustrates an embodiment of methods for registering (calibrating) and tracking;

FIG. 3 illustrates an embodiment of methods for registration of a medical image using detected coordinates of radio-opaque fiducial markers;

FIG. 4A illustrates an embodiment of the use of calibration frames with the guidance system;

FIG. 4B illustrates an embodiment of the use of calibration frames with the guidance system;

FIG. 4C illustrates an embodiment of the characterization of calibration frames with the guidance system;

FIG. 4D illustrates an embodiment of the use of calibration frames with the guidance system;

FIG. 4E illustrates an embodiment of the use of calibration frames with the guidance system;

FIG. 5A illustrates an embodiment of an dynamic reference array, comprising a modified mount with a clamping mechanism;

FIG. 5B illustrates an embodiment of an dynamic reference array, comprising a modified mount with a clamping mechanism;

FIG. 5C illustrates an embodiment of an dynamic reference array, comprising a modified mount with a clamping mechanism;

FIG. 6A illustrate an embodiment of clamping mechanism actuation on a spinous process;

FIG. 6B illustrate an embodiment of clamping mechanism actuation on a spinous process;

FIG. 7A illustrate an embodiment of clamping mechanism actuation on a spinous process;

FIG. 7B illustrate an embodiment of clamping mechanism actuation on a spinous process;

FIG. 8A illustrates a clamping mechanism modified with a dynamic reference array including a temporary marker skirt;

FIG. 8B illustrates a clamping mechanism modified with a dynamic reference array with the temporary marker skirt detached;

FIG. 9A illustrates a dynamic reference array in a lowered position;

FIG. 9B illustrates a dynamic reference array in a upper position

FIG. 10A illustrates a dynamic reference array in a lowered position with an alternate tracker in a static position;

FIG. 10B illustrates a dynamic reference array in a lowered position with an alternate tracker in an static position;

FIG. 11A illustrates two posts that may be used to anchor the dynamic reference array to bone;

FIG. 11B illustrates an interior view of an open clamp used to secure the two posts in a selected position; and

FIG. 11C illustrates a closed clamp, securing the two posts from movement.

### DETAILED DESCRIPTION

Dynamic reference arrays, herein referred to as “DRAs”, are rigid bodies that are temporarily attached to the patient during a navigated surgical procedure. Their purpose may be to allow 3D localization systems to track the positions of tracking markers that are embedded in the DRA, and thereby track the real-time position of relevant anatomy. A step that may be needed before such tracking may provide useful data may be to register the anatomy such that the transformation from the coordinate system of the anatomy (for example, a 3D CT scan volume) to the coordinate system of the tracking system may be defined. Registration methods are briefly discussed below.

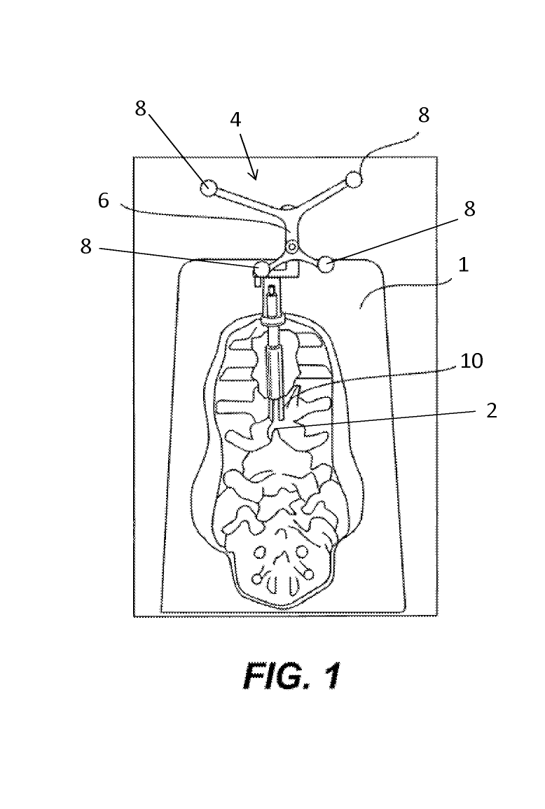

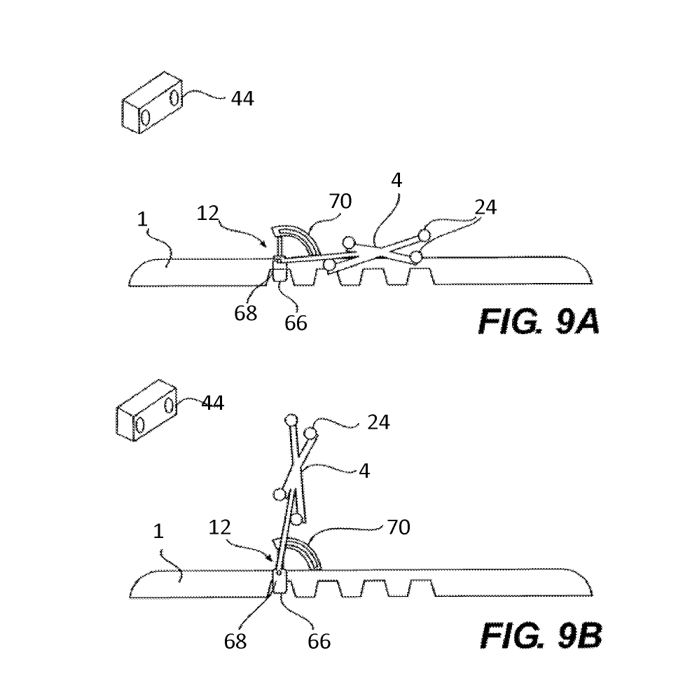

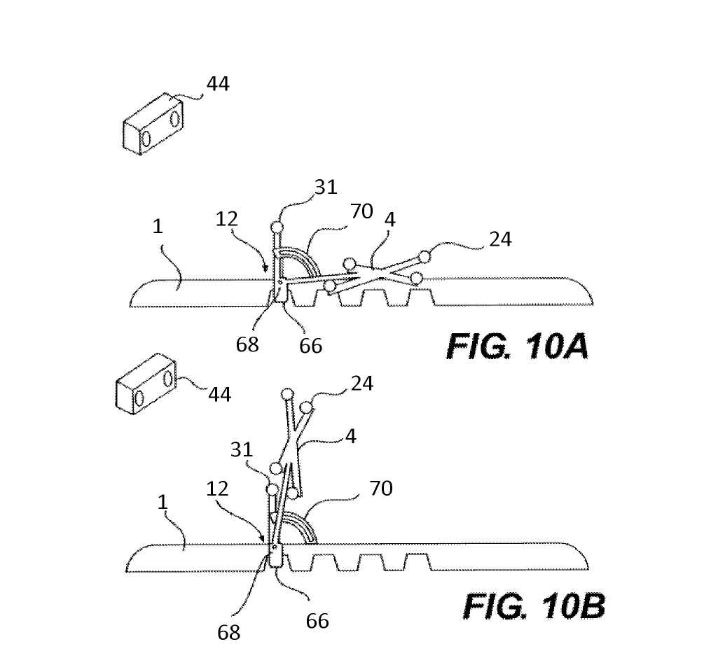

FIG. 1 illustrates a representation of a spinous process 2 within a model of a patient 1. A DRA 4 may be attached to spinous process 2. This particular DRA 4 comprises a scaffold 6, a plurality of tracking markers 8 and a clamp mechanism 10. Scaffold 6 and clamp mechanism 10 may be in a single structure or connected by an angular adjustment swivel feature 12, as illustrated in FIGS. 9A, 9B, 10A, 10B. Swivel feature 12 may include a hinge, ball-and-socket or other suitable joint to allow the orientation of scaffold 6 to be adjusted while DRA 4 remains rigidly clamped or fastened to bone. Tracking markers 8 may be positioned at any suitable location on scaffold 6. Tracking markers 8 may be any lightweight device whose 3D position may be sensed accurately using a detector. For example, tracking markers 8 may be reflective spheres tracked using stereophotogrammetry by two or more optical cameras, infrared-emitting diodes tracked using stereophotogrammetry by two or more optical cameras, magnetic sensors capable of detecting the position within a magnetic field, and/or radiofrequency emitters whose position may be sensed through time-of-flight to radiofrequency receivers in fixed known positions around the room. The positions of tracking markers 8 relative to their tracking system may be adjusted using swivel feature 12. For example, the positions of reflective spheres on DRA 4 may be adjusted using swivel feature 12 so that there may be better line of sight from the cameras to the fixture.

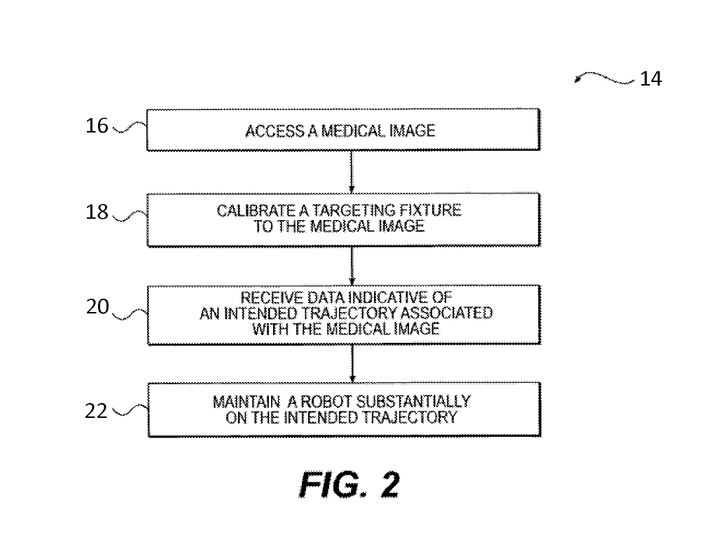

FIG. 2 illustrates an algorithm, which may be used in a registration method. As illustrated, a registration method 14 begins with block 16. Block 16 may comprise accessing (e.g., receiving, retrieving, or otherwise acquiring) a medical image. As described herein, the medical image may be a 3D anatomical image scan including, but not limited to a CT scan, a magnetic resonance imaging scan (hereinafter referred to as an “MRI scan”), three-dimensional fluoroscopy scan, and/or other anatomical scan. It should be appreciated that any 3D anatomical scan may be utilized with a surgical robot, not illustrated, and may be within the scope of the present invention. In some embodiments, at block 18, registration method 14 may further comprise calibrating a DRA 4 to the medical image. In some embodiments, the calibration may be semi-automated or automated. In some embodiments, at block 20, the registration method 14 may further comprise receiving data indicative of an intended trajectory associated with the medical image. In some embodiments, at block 22, after registration is complete, registration method 14 may further comprise maintaining a robot substantially on the intended trajectory. In some embodiments, a control platform (not illustrated) may adjust movement of the robot in order to substantially maintain the intended trajectory.

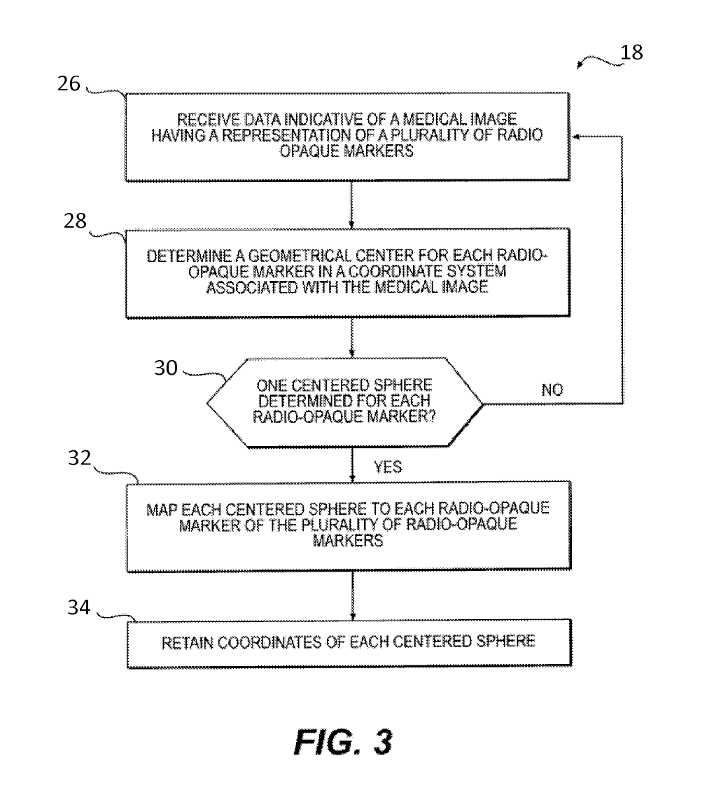

As illustrated in FIG. 3, block 18, to register a DRA 4 to the medical image a semi-automated calibration method may be implemented. Starting with block 26, data indicative of a medical image having a representation of a plurality of radio-opaque fiducial markers 24 (e.g., fiducial markers 24 on FIG. 1) may be received. In one embodiment, as described herein, such plurality may contain four fiducial markers 24. In some embodiments, at block 28, a geometrical center for each fiducial marker 24 may be determined in a coordinate system associated with the medical image.

In some embodiments, image thresholding may be utilized to define one or more edges of each fiducial marker 24 and a geometrical center thereof. Thresholding refers to an image processing technique in which pixel intensity within a two dimensional (2D) region may be monitored. For example, the x, y positions (for instance expressed in mm) of pixels of an intensity that reach a predetermined value may be retrieved. Stated similarly, the threshold refers to the transition pixel intensity from light to dark. In some embodiments, on 2D slices of the medical image, fiducial marker 24 may appear light and the adjacent space (such as tissue or air) may appear dark. In some embodiments, displaying pixels that satisfy a thresholding criterion at an intensity encountered at the edge of a fiducial marker 24 may yield a largely circular trace outlining the marker on the medical image. Since in some embodiments, fiducial markers 24 may be spherical, a method for finding the center of fiducial marker 24 in a 2D view may include firstly restricting the 2D view to a sampling region with the high-intensity image of the sphere toward the center of the region and pixels of lower intensity toward the outer edges of the region. Secondly, the method may include finding the mean x threshold position (e.g., the maximum x coordinate of pixels satisfying the threshold criterion plus minimum x coordinate of pixels satisfying the threshold criterion divided by two), and finding the mean y threshold position using a similar method.

In some embodiments, the center of the sphere may be found by determining 2D centers of slices through the same fiducial marker 24 in two orthogonal views. For example, in some embodiments, the method may include finding mean x and mean y from an xy slice, then finding mean x and mean z from an xz slice to get a mean x, y, and z axis coordinate representing the center of fiducial marker 24. Further, upon or after the mean x, mean y, and mean z are found, new xy and xz slices may be evaluated again and the maximum and minimum x, y, and z threshold values may be again determined to evaluate the dimensions of the thresholded object in each view. It may be appreciated from this method that in some embodiments, a non-spherical object of high intensity, such as a small process of cortical bone extending away from the side of the spine, may fail to satisfy (1) a condition where there may be high intensity near the middle of the region, but low intensity all around, since the process may extend out of the region in one or more directions; or (2) a condition where the dimensions in x, y, and z of the centered object do not match each other (e.g., non-spherical case).

As illustrated in FIG. 3, in some embodiments, at block 30, it is ascertained if one centered sphere is determined for each fiducial marker 24 for the fixture being calibrated. In some embodiments, when at least one such sphere is not determined, or identified, the threshold setting is adjusted and flow is directed to block 26. In some embodiments, at block 32, each centered sphere is mapped to each fiducial marker 24 of the plurality of fiducial markers 24. As illustrated, in some embodiments, block 32 may represent a mapping action which, in some embodiments, may comprise implementing a sorting process to establish a specific centered sphere is associated with a specific one of the plurality of fiducial markers 24.

In some embodiments, a plurality of fiducial markers 24 may contain four fiducial markers 24 (represented, for example, as OP1, OP2, OP3, and OP4). In some embodiments, the sorting process may map each one of four centered fiducial markers 24 to one of OP1, OP2, OP3, or OP4. In some embodiments, the sorting process may distinguish a specific fiducial marker 24 by measuring inter-marker distances from mean positions of the four unidentified fiducial markers 24, and comparing such distances to extant inter-marker distances (for example, those that are pre-measured and retained in memory, such as mass storage device) for each fiducial marker 24 on a marker fixture. In some embodiments, fiducial markers 24 on DRA 4 may be placed asymmetrically, each fiducial marker 24 may be identified from a unique set of inter-marker distances corresponding to such fiducial marker 24. For example, in some embodiments where the sum of inter-marker distances of one unknown fiducial marker 24 relative to the other threes fiducial markers 24 measured from the medical image is D, a single physical fiducial marker 24 (one of OP1, OP2, OP3, or OP4) may have a matching inter-marker distance sum within a specified tolerance (such as ±1 mm) of D. In some embodiments, at block 34, coordinates of each centered sphere may be retained (for example in memory of a computer platform).

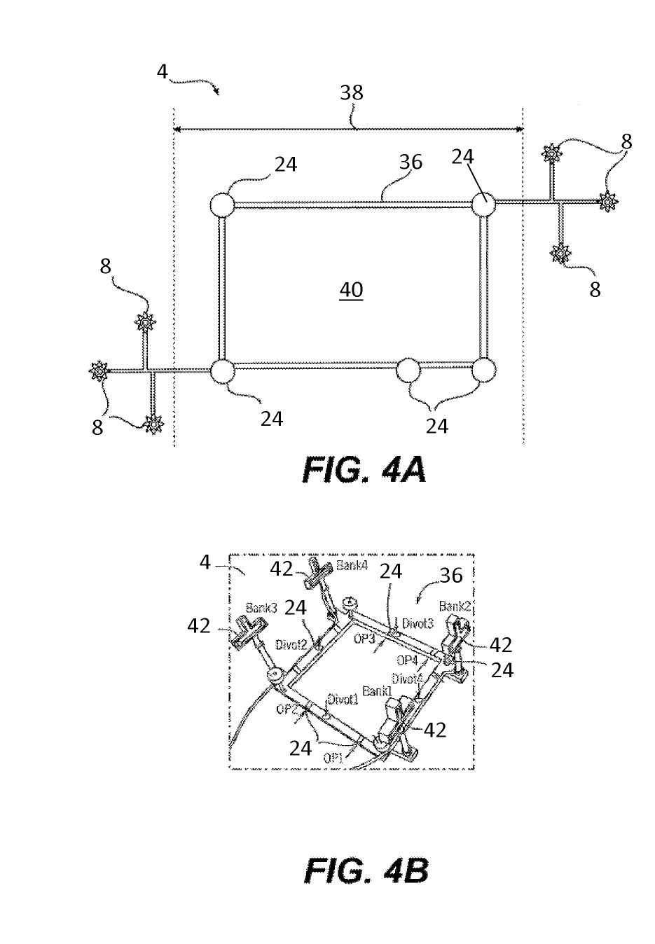

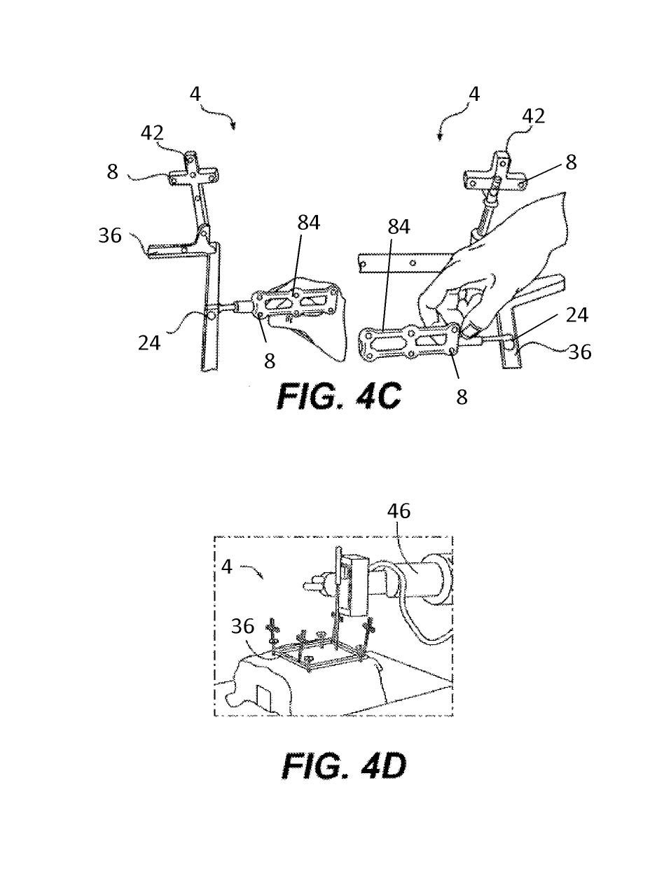

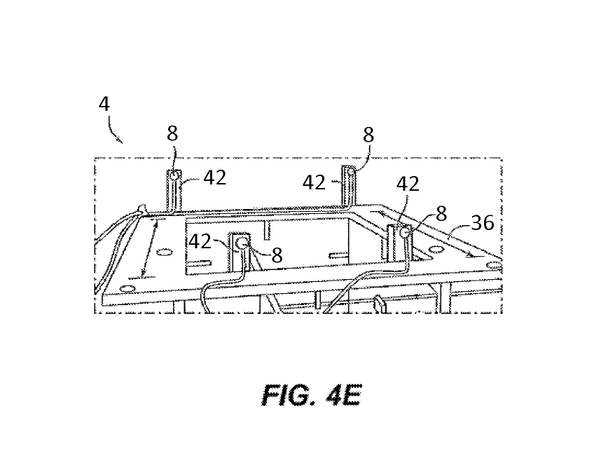

FIGS. 4A-4E illustrate alternate guidance systems used with a surgical robot system. In embodiments, a surgical robot system, not illustrated, may comprise a DRA 4 for use with a guidance system. In some embodiments, one DRA 4 comprises a calibration frame 36, as illustrated in FIGS. 4A-4E. A calibration frame 36 may be used in connection with many invasive procedures. For example, calibration frame 36 may be used in thoracolumbar pedicle screw insertion in order to help achieve a more accurate trajectory position. In some embodiments, the use of calibration frame 36 may simplify the calibration procedure. In some embodiments of the invention, calibration frame 36 may be temporarily affixed to the skin of a patient 1 (e.g., FIG. 1) surrounding a selected site for a medical procedure, and then the medical procedure may be performed through a window defined by calibration frame 36.

As illustrated in FIGS. 4A and 4B, in some embodiments of the invention, calibration frame 36 may comprise a combination of fiducial markers 24 and tracking markers 8. In some embodiments, fiducial markers 24 may be located within CT scan region 38, and tracking markers 8 may be located outside of CT scan region 33. In some embodiments, a surgical field 40 (i.e., the area where the invasive procedure will occur) may be located within the perimeter created by fiducial markers 24. In some embodiments, the actual distances of fiducial markers 24 and tracking markers 8 relative to each other may be measured from a high-precision laser scan of calibration frame 36. Additionally or alternatively, in some embodiments, the actual relative distances may be measured by actively measuring the positions of tracking markers 8 while nearly simultaneously or simultaneously pointing with a pointing device, such as a conventional digitizing probe, to one or more locations on the surface of the fiducial markers 24. In certain embodiments, digitizing probes may comprise tracking markers 8 embedded in a rigid body 40 and a tip extending from rigid body 40.

In some applications, to establish the spatial relationship between tracking markers 8 and fiducial markers 24, a conventional digitizing probe, such as a 6-marker probe, embedded with tracking markers 8 in a known relationship to the probe’s tip (see for example FIG. 4C) may be used to point to each fiducial markers 24. In some embodiments, the probe may point to locations on two opposite surfaces of spherical fiducial markers 24 while recording the position of the probe tip and tracking markers 8 on calibration frame 36 simultaneously. Then, the average position of the two surface coordinates may be taken, corresponding to the center of the sphere. An image of a robot 46, which may be used with DRA 4 is illustrated in FIG. 4D. For placement of conventional surgical screws, a biopsy, injection, or other procedures, in some embodiments, robot 46 may work through the window formed by calibration frame 36. During a surgical procedure, in some embodiments, the working portal is kept on the interior of calibration frame 36 and fiducial markers 24 on the exterior of calibration frame 36 may improve accuracy over a system where fiducials are mounted away from the area where surgery is being performed. Without wishing to be bound by theory, simulation, and/or modeling, it is believed that a reason for improved accuracy is that optimal accuracy of tracking markers 8 may be achieved if tracking markers 8 are placed around the perimeter of calibration frame 36 being tracked.

Further embodiments of calibration frame 36 are illustrated in FIG. 4E. This embodiment is simplified to make it less obstructive to the surgeon. In some embodiments, calibration frame 36 may comprise four tracking markers 8 having a lower profile than tracking markers 8 described above and depicted in FIGS. 4A-4D. For example, calibration frame 36 may comprise a plurality of upright posts 42 that are angled away from calibration frame 36 by about 10º. In some embodiments, tracking markers 8 are mounted on posts 42 that are angled back by 10º, and this angulation keeps tracking markers 8 facing toward the cameras despite the patient being horizontal.

Moreover, in some embodiments, a pair of the tracking markers 8 positioned on the front of calibration frame 36 may be configured to have less chance of obscuring the pair of tracking markers 8 positioned on the rear of calibration frame 36. For example, posts 42 that are farthest away from the camera or farthest from a detection device of a tracking system 44, as illustrated in FIGS. 9A, 9B, 10A, and 10B, may be taller and spaced farther laterally than posts 42 closest to the camera.

In additional embodiments, calibration frame 36 may comprise fiducial markers 24 that comprise radio-opaque properties for detection by a medical imaging scanner, and tracking marker 8 properties, allowing fiducial markers 24 to be visible by cameras or otherwise detectable by real-time tracking system 44. In some embodiments, relationship between fiducial marker 24 and tracking markers 8 may not need to be measured or established because fiducial marker 24 contains properties of both types of detection. Therefore, in some embodiments, as soon as the position is determined from the CT scan (or other imaging scan), the spatial relationship between robot 46 and anatomy of patient 1 may be defined.

In other embodiments, DRA 4 may comprise a flexible roll configuration. In some embodiments, DRA 4 may comprise three or more radio-opaque fiducial markers 24 that define a rigid outer frame and nine or more tracking markers 8 embedded in a flexible roll of material. As described earlier, fiducial markers 24 may be visible on CT scans and/or other medical diagnostic images, such as MRI, or reconstructions from O-arm or Iso-C scans, and their centroids may be determined from the 3D image. Tracking markers 8 may include tracking markers 8 that have 3D coordinates that are detectable in real-time using cameras or other means. Some embodiments may utilize tracking marker systems based on reflective optical systems, infrared-emitting marker systems, electromagnetic systems, or a Local Positioning System (“LPS”).

In some embodiments, DRA 4 may be an adherable fixture, configured for temporary attachment to the skin of a patient 1. For example, in some embodiments, DRA 4 may be temporarily adhered to the patient 1 during imaging, removed, and then subsequently reattached during a follow-up medical procedure, such as a surgery. In some embodiments, DRA 4 may be applied to the skull of a patient 1 for use in placement of electrodes for deep brain stimulation. In some embodiments, this method may use a single scaffold 6, or two related scaffold 6. In this instance, the two related scaffolds 6 may share the same surface shape. However, one scaffold 6 may be temporarily attached at the time of medical image scanning, and may include fiducial markers 24 (but not tracking markers 8), and second scaffold 6 may be attached at the time of surgery, and may include tracking markers 8 (but not fiducial markers 24).

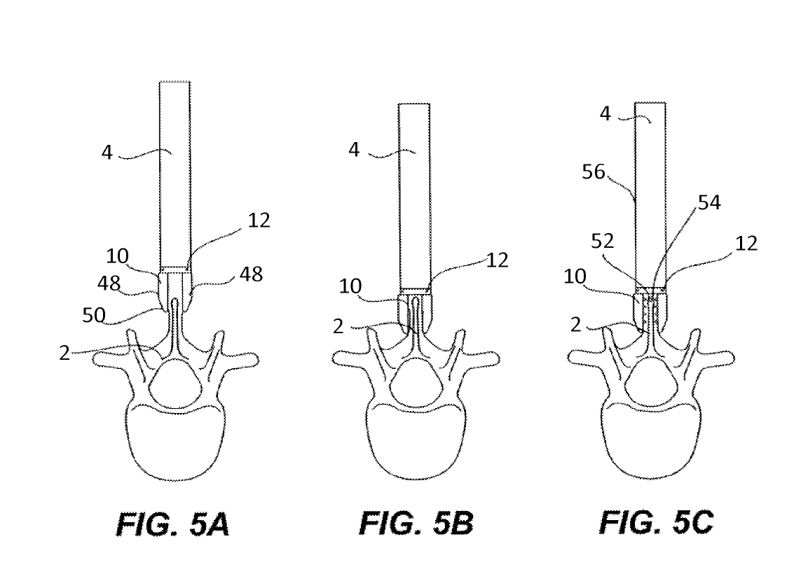

In embodiments of the invention, DRA 4 may comprise a conventional clamping mechanism 10 (e.g., FIG. 1) for securely attaching DRA 4 to patient 1. For example, in some embodiments, DRA 4 may be configured to clamp to spinous process 10 of a patient 1 after the surgeon has surgically exposed spinous process 10. FIGS. 5A-5C shows the lower support of a DRA 4 mounted to spinous process 10 in the lumbar spine of a patient 1 in accordance with some embodiments.

In embodiments, during use of a DRA 4 having a conventional clamping mechanism 10 with image guidance, the relationship between fiducial markers 24 and the bony anatomy of patient 1 may be established using a registration process wherein known landmarks are touched with a digitizing probe at the same time that tracking markers 8 on the tracker are visible. In some embodiments of the invention, the probe itself may have a shaft protruding from a group of fiducial markers 24, or tracking markers 8, thereby permitting tracking system 44, illustrated in FIGS. 9A, 9B, 10A, and 10B, to calculate the coordinates of the probe tip relative to fiducial markers 24, or tracking markers 8.

In embodiments, clamping mechanism 10 of DRA 4 may be configured for clamping to spinous process 10, or may be configured for anchoring to bone of patient 1 such that DRA 4 may be substantially stationary and not easily moved. In some further embodiments, DRA 4 may comprise at least three tracking markers 8 and distinct fiducial markers 24 that are detected on the CT or other 3D image, preferably near clamp 16 (to be close to bone). In embodiments, tracking markers 8 themselves may be configured to be visualized accurately on CT or other 3D image. In certain embodiments, the portion of scaffold 6 containing a fiducial markers 24 may be made to be detachable to enable removal from scaffold 6 after the 3D image is obtained. In embodiments, a combination of fiducial markers 24 and tracking markers 8 may allow tracking with robot 46 in the same way that is possible with the frame-type DRA 4 described above.

As a further illustration of a procedure using an alternate guidance system, in some embodiments, the steps of an open screw insertion procedure utilizing an optical guidance system is described. In some embodiments, after surgical exposure, a DRA 4 comprising a small tree of tracking markers 8, for example tracking markers 8, may be attached to a bony prominence in the area of interest. In some embodiments, conventional calibration procedures for image guidance may be utilized to establish the anatomy relative to tracking system 44 and medical images. For another example, DRA 4 may contain rigidly mounted, substantially permanent or detachable fiducial markers 24 that may be imaged with a CT scan. In some embodiments, the calibration procedures consistent with those stated for calibration frame 36 may be utilized to establish the anatomy relative to robot 46 and the medical image.

In embodiments, an extension to the methods for reconstructing tracking markers 8 is to use multiple ambiguous synchronized lines of sight via multiple cameras tracking the same tracking markers 8. For example, two or more cameras may be set up from different perspectives focused on tracking markers 8 on DRA 4 or robot 46. In embodiments, one camera unit may be placed at the foot of a patient’s bed, and another may be attached to robot 46. In some embodiments, another camera unit may be mounted to the ceiling. In embodiments, when all cameras substantially simultaneously view tracking markers 8, coordinates may be transformed to a common coordinate system, and the position of any of tracking markers 8 may be considered to be the average (mean) of that marker’s three dimensional position from all cameras used. In embodiments, even with extremely accurate cameras, an average may be needed because with system noise, the coordinates as perceived from different cameras may not be exactly equal. However, when one line of sight is obscured, the lines of sight from other cameras (where tracking markers 8 may still be viewed) may be used to track robot 46 and DRA 4. In embodiments, to mitigate twitching movements of robot 46 when one line of sight is lost, it is possible that tracking marker 8 positions from the obscured line of sight may be reconstructed using methods as previously described based on an assumed fixed relationship between the last stored positions of tracking markers 8 relative to the unobstructed lines of sight. Further, in embodiments, the position of tracking marker 8 from camera one relative to its position from camera two may be stored; then if camera one is obstructed, and until the line of sight is restored, this relative position may be recalled from computer memory (for example in memory of a computer platform) and a reconstruction of tracking marker 8 from camera one may be inserted based on the recorded position of tracking marker 8 from camera two. In some embodiments, the method may compensate for temporary obstructions of line of sight such as a person standing or walking in front of one camera unit.

In embodiments, instead of a DRA 4 consisting of a combination of fiducial markers 24 and tracking markers 8, it is possible to register a primary DRA 4 through an intermediate registration of another temporary DRA 4. For example, in some embodiments, an example of such a calibration method may include attaching a temporary rigid plate, not illustrated, that contains fiducial markers 24, open mounts (such as snaps, magnets, Velcro, or other features) to which tracking markers 8 may at any time be attached in a known position. The method may then include scanning the subject (using for example CT, MRI, etc.), followed by attaching a primary DRA 4, not illustrated, such as those described earlier or other DRA with three or more tracking markers 8 rigidly affixed to the anatomy of a patient 1, and then attaching tracking markers 8 to the temporary DRA 4 in the known positions dictated by the snaps, magnets, velcro, etc. This primary DRA 4 may not require any fiducial markers 24 because registration is performed through the temporary DRA’s 15 fiducial marker 24 positions. In some embodiments, a further step may include activating cameras to read the position of the primary DRA 4 affixed to the anatomy of patient 1 at the same time as second temporary DRA 4. This step establishes the position of tracking markers 8 on the temporary DRA 4 relative to the positions of tracking markers 8 on the primary DRA 4, because the position of fiducial markers 24 on the temporary DRA 4 are known relative to tracking markers 8 on the temporary DRA 4, establishing the position of the anatomy relative to tracking markers 8 on the primary DRA 4. After establishing position, the temporary DRA 4 may be removed, including its tracking markers 8 and fiducial markers 24. These markers are no longer needed because registration has been transferred to the tracking markers 8 on the rigidly affixed primary DRA 4.

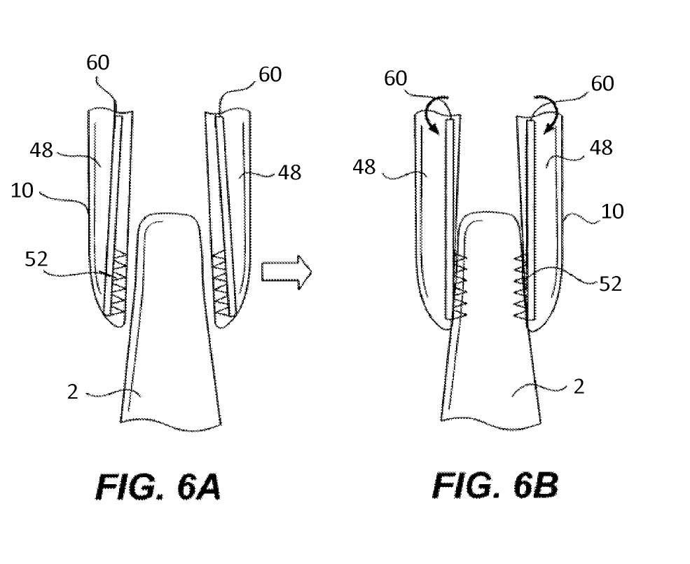

In embodiments, as illustrated in FIGS. 5A-5C, may comprise a modification to DRA 4 allowing DRA 4 to slide a clamping mechanism 10 over spinous process 2 without full exposure of spinous process 10. As illustrated, clamping mechanism 10 may comprise clamps 48 including at least one beveled edge 50, and clamp teeth 52. During an operation, a surgeon may make a stab incision in the midline and then slide clamps 48 of clamping mechanism 10 down along the sides of spinous process 2, pushing tissue away as the tip of clamping mechanism 10 is advanced. In some embodiments, as illustrated in FIGS. 6A and 6B, the leading edge of clamping mechanism 10 may be beveled (see leading beveled edges 50 of each clamp 48 of clamping mechanism 10), and have a shape similar to a periosteal elevator. This shape may allow clamping mechanism 10 to separate the muscle tissue from the bony spinous process 2 as it is advanced. In some embodiments, leading beveled edges 50 of clamping mechanism 10 may be electrified to enable it to more easily slide through muscle and connective tissues to prevent excessive bleeding.

In some embodiments, a mechanism activated from farther back on the shaft (for example a turn screw, or conventional spring, etc.) may be activated to deploy clamp teeth 52 on clamps 48. The same mechanism or another mechanism may close and compress clamps 48 together to firmly secure clamping mechanism 10 to spinous process 2 (see FIGS. 5B-5C). Additionally, in some embodiments, a screw 54 aligned with a handle 56 may deploy by threading into spinous process 2 (see for example, FIG. 5C).

The embodiments as described above and illustrated in FIGS. 5A-5C may be especially well suited to percutaneous pedicle screw-rod surgery because the hole made for mounting clamping mechanism 10 may also be used as the hole for inserting the conventional rod to interconnect the conventional pedicle screw heads. Further, the embodiments as described above and illustrated in FIGS. 5A-5C may also be useful for mounting a marker tree (for other bony prominences, such as transverse processes, long bones, skull base, or others).

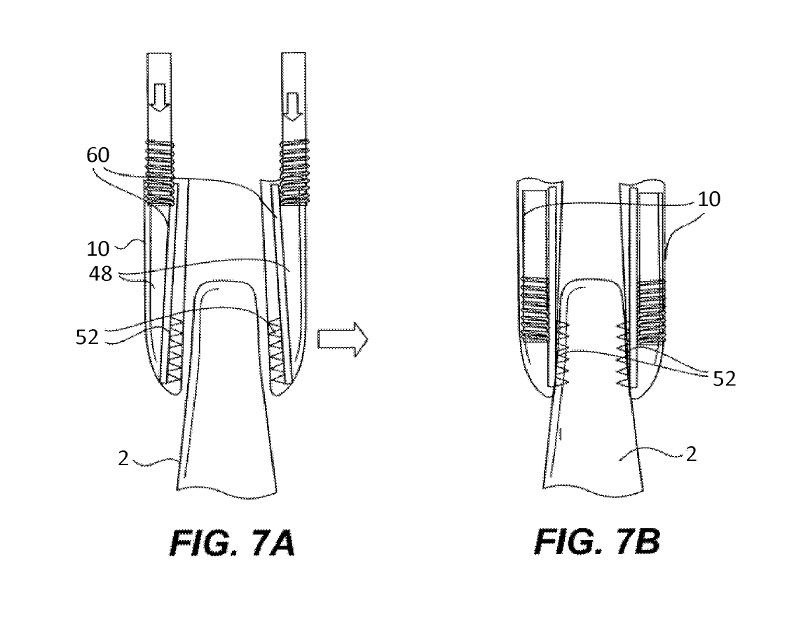

FIGS. 6A-6B and 7A-7B illustrate embodiments of clamping mechanism 10 actuation on a spinous process 2 in accordance with some embodiments. In some embodiments, the mechanism for deploying clamp teeth 52 may be comprise a hollow cavity 58 containing clamp teeth 52 that are to one side of hollow cavity 58 during insertion, but may be forced toward the opposite side when the mechanism is deployed, such that the embedded teeth penetrate the bone (see the illustration of penetrated clamp teeth 52 in FIGS. 7A and 7B).

FIGS. 7A-7B illustrate an alternative embodiment of clamping mechanism 10 actuation on a spinous process 2. As illustrated, the groups of clamp teeth 52 may be attached to rods 60 that run down hollow cavity 58. Rods 60 may pivot farther up handle 56 (pivot point not pictured) and force clamp teeth 52 together. For example, in embodiments, rods 60 may be driven into hollow cavity 58 on the side away from the bone, forcing clamp teeth 52 against and into the bone (for example, see the penetrated clamp teeth 52 in FIG. 7B).

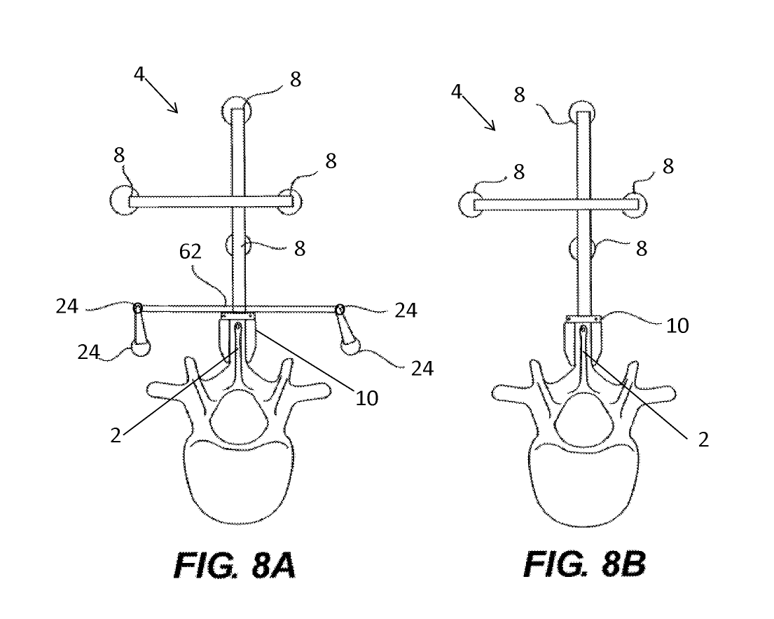

As described above, fiducial markers 24 may be present in a CT scan of the anatomy. However, it may be desirable to crop CT scans as close as possible to the spine to improve resolution. In some embodiments, instead of using fiducial markers 24 near where tracking markers 8 are located, an alternative may be to have a rigid extension containing fiducial markers 24 that may be temporarily attached near spinal process 2 when the scan is taken. In some embodiments, clamping mechanism 10 may be coupled with, or otherwise modified with a DRA 4. For example, FIGS. 8A-8B illustrate a clamping mechanism 10 modified with a DRA 4 including a temporary marker skirt 62 in accordance with at least one embodiment of the disclosure, and FIG. 8B illustrates a clamping mechanism 10 modified with a DRA 4 as illustrated in FIG. 8A with temporary marker skirt 62 detached in accordance with at least one embodiment of the disclosure. As illustrated, temporary marker skirt 62 may include fiducial markers 24 in a temporary “skirt” around the base of clamping device 16. The design of temporary marker skirt 62 and clamping device 16 may be such that fiducial markers 24 in skirt 62 have known locations relative to tracking markers 8 for tracking that are farther away. Once the scan is taken, fiducial markers 24 may not be needed. Therefore, in embodiments, by depressing a conventional release, temporary marker skirt 62 may be removed, so it will not be in the way of the surgeon (see for example FIG. 8B).

In embodiments, as illustrated in FIGS. 9A-9B, a DRA 4 may have a swiveling feature 12 that enables the position of some or all tracking markers to be shifted after the scan is captured. One benefit of this feature is that it allows the radio-opaque markers to be positioned in the location that is most appropriate for the CT scan—near the anatomy, close to the skin, as illustrated in FIG. 9A. For example, the DRA 4 may be positioned generally flat (e.g., angled about 10° or less) along the patient. Then after the CT scan, it allows the user to swing the DRA 4 upwards, placing fiducial markers 24 away from surgery, while simultaneously positioning the tracking markers in a position that is appropriate for tracking, as illustrated in FIG. 9B. For example, the DRA 4 may be moved into an upright position. Swiveling may enable fiducial markers 24 to be mounted nearer to the tracking markers than other designs, thereby making the entire DRA 4 more compact. Swiveling may allow the same markers to double as both radio-opaque fiducial markers 24 and tracking markers 8. For example, reflective tracking spheres may be manufactured with a metal or ceramic core that is radio-opaque.

An additional benefit and feature of the swiveling feature 12 may be that one or more of fiducial markers 24, or tracking markers 8, may be made to move relative to the other tracking markers during swiveling, altering the rigid body relationship of the tracking markers, as illustrated in FIG. 10A-10B. In other words, the relative positioning of one or more of fiducial markers 24, or tracking markers 8, may shift relative to the configuration that is stored in computer memory and compared during any given tracking frame. When the rigid body array spacing is altered, it is possible for software to determine whether the array is down or up automatically. This feature prevents the user from inadvertently trying to track and navigate before DRA 4 is swiveled into the correct position. Additionally, one or more fiducial markers 24 may be made to move relative to other fiducial markers 24 during swiveling, meaning two discrete rigid body arrangements may be observed on the CT scan, allowing software to automatically detect whether the array was up or down during the scan. This may prevent the user from inadvertently trying to track and navigate before DRA 4 is in the correct position. FIGS. 10A-10B illustrates a configuration where one fiducial marker 24, or tracking marker 31, may not swivel with a plurality of additional fiducial markers 24, and/or tracking markers 8.

To properly swivel, DRA 4 may comprise a base 66 which may be part of the clamping mechanism 10. Base 66 may be attached at a connection point 68 to DRA 4, with fiducial markers 24 and/or tracking markers 8, by any suitable means. Suitable means may be, but is not limited to a ball joint, a hinge, a slide, or any combination thereof. Furthermore, DRA 4 may swivel in any direction in relation to base 66. This may allow the structure to move from side to side, up or down, diagonally, or any combination thereof. Movement of DRA 4 in relation to base 66 may further be aided by a guide 70 or external member. In embodiments, not illustrated, a DRA 4 may swivel at more than one location located on base 66 or DRA 4.

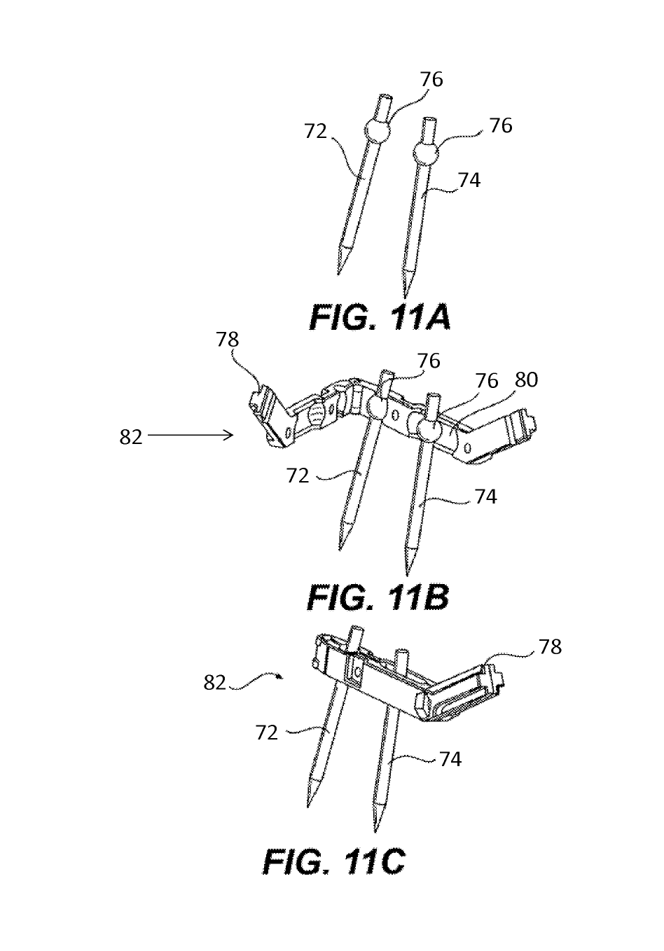

In some cases, it may not be possible to mount a clamp mechanism 10. For example, surgery may be intended on a region other than the spine, or surgery may be intended in cases in which the patient previously had the spinous process removed. As illustrated in FIGS. 11A-11C, an alternate method for mounting a DRA 4 rigidly to the bone of patient 1 may consist of two posts 72 and 74 onto which a DRA 4 clamps. It is contemplated that two posts 72 and 74 may have a spherical feature 76 to constrain some translational and rotational degrees of freedom, enabling locking of DRA 4 in a rigidly fixed and repeatable position by clamp 78. Clamp 78, in embodiments, may serve as a base in which swivel feature 12 may seat. For this two-post method, as illustrated in FIGS. 11A-11C, the interior of clamp 78 secured to a post 72 may have a socket-shaped feature with a flared through-hole. Clamp 78 may constrain all translation of the socket piece while still allowing it to rotate. Part of DRA clamp 78 around post 76 may have an elongated socket 80 with a racetrack shaped through-hole. This feature may constrain all rotation of DRA 4 and translation of the other pin in most directions. This configuration may allow two-post DRA 82 to be clamped into a unique orientation and position then removed and repeatedly re-attached to the same position.

In an embodiment, where mounting post 72 to a different vertebra than post 74, this configuration does not prevent translation of post 72 toward or away from post 74, in which case post 74 may slide inside elongated socket 80. It may be possible to tighten tolerances so that when two-post DRA 82 is clamped around post 74, friction disallows such translation. Or such shifting may be monitored through surveillance markers.

Although the present invention and its advantages have been described in detail, it should be understood that various changes, substitutions and alterations may be made herein without departing from the spirit and scope of the invention as defined by the appended claims.

What is claimed is:

1. A method of using a dynamic reference array including a plurality of markers, the method comprising:
attaching the dynamic reference array to a patient at a base and a connection point, wherein the plurality of markers are both radiopaque and optically reflective;
scanning the patient while the plurality of markers of the dynamic reference array are in a first position;
registering the dynamic reference array to the patient's anatomy based on the scanning;
after the registration, moving at least one marker of the plurality of markers to a second position while the entire dynamic reference array remains attached to the patient, wherein the dynamic reference array is movable relative to the base at the connection point and moving the at least one marker includes moving the marker without detaching the marker from the dynamic reference array; and
determining, using an image guidance software, whether the plurality of markers are in the first or second position for purposes of preventing inadvertent tracking and navigation.

2. The method of claim 1, wherein the step of moving includes moving the at least one marker of the plurality of markers away from a surgical site of the patient.

3. The method of claim 1, wherein the step of moving includes moving the at least one marker of the plurality of markers away from a surgical site of the patient, the method further comprising:
determining, using an image guidance software, whether the plurality of markers are in the first or second position for purposes of preventing inadvertent tracking and navigation.

4. The method of claim 1, wherein attaching the dynamic reference array to the patient comprises clamping the dynamic reference array onto a spinous process of the patient.

5. The method of claim 1, wherein attaching the dynamic reference array to the patient comprises securing at least one post into the patient.

6. The method of claim 1, wherein each marker includes a radio-opaque core.

7. The method of claim 1, wherein the plurality of markers are both radio-opaque and infrared reflective.

8. The method of claim 1, wherein moving at least one of the markers of the plurality of markers to a second position includes moving the at least one marker of the plurality of markers with respect to the other markers of the plurality of markers.

9. The method of claim 1, wherein:
scanning includes scanning the patient with an imaging device;
registering includes registering the dynamic reference array to the patient's anatomy based on the plurality of markers contained in the scanned image.

10. The method of claim 9, wherein the plurality of markers are both radio-opaque visible in the scanned image and infrared reflective visible in the camera tracking system.

11. A method of using a dynamic reference array including a plurality of markers, the method comprising:
attaching the dynamic reference array to a patient bone, wherein the plurality of markers are both radiopaque and optically reflective;
scanning the patient with a medical imaging device while the plurality of markers of the attached dynamic reference array are in a first position;
registering the dynamic reference array to the patient's anatomy based on the plurality of markers contained in the scanned medical image; and
after the registration, moving at least one marker of the plurality of markers to a second position relative to the other markers of the plurality of markers while the entire dynamic reference array remains attached to the patient, wherein the dynamic reference array swivels relative to the base, wherein the moved markers in the second position are positioned to be tracked by a camera tracking system and moving the at least one marker includes swiveling the marker without detaching the marker from the dynamic reference array
determining, using an image guidance software, whether the plurality of markers visible in a camera tracking system are in the first or second position for purposes of preventing inadvertent tracking and navigation.

12. The method of claim 11, further comprising:
preventing tracking of the plurality of markers of the dynamic reference array based on the position of the plurality of markers.

13. The method of claim 11, wherein the step of moving includes moving the at least one marker of the plurality of markers away from a surgical site of the patient.

14. The method of claim 11, wherein the step of moving includes moving the at least one marker of the plurality of markers away from a surgical site of the patient, the method further comprising:
determining, using an image guidance software, whether the plurality of markers are in the first or second position for purposes of preventing inadvertent tracking and navigation.

15. The method of claim 11, wherein each marker includes a radio-opaque core.

16. The method of claim 11, wherein the plurality of markers are both radio-opaque visible in the scanned image and infrared reflective visible in the camera tracking system.

17. The method of claim 16, wherein the step of determining includes determining whether the plurality of markers are in the first or second position based on an electronic image captured by the camera tracking system for purposes of preventing inadvertent tracking and navigation.
